(19) United States
(12) Patent Application Publication
(10) Pub. No.: US 2026/0325772 A1
(43) Pub. Date: Oct. 22, 2026

(54) Rapid Determination of Transpulmonary Pressure in a Patient Connected to a Breathing Apparatus

(71) Applicant: The Lung Barometry Sweden AB, Viken (SE)

(72) Inventor: Ola Stenqvist, Viken (SE)

(21) Appl. No.: 19/491,061

(22) PCT Filed: Jun. 2, 2024
(86) PCT No.: PCT/EP2024/065137

§ 371 Date: Dec. 8, 2025

(30) Foreign Application Priority Data
Jun. 8, 2023 (SE) 2350700-7

(51) Int. Cl. A61M 16/00

(52) U.S. Cl. CPC A61M 16/022; A61M 2205/3344; A61M 2205/3379; A61M 2205/50; A61M 2230/40

(57) ABSTRACT

In particular, a method is disclosed for determination of End inspiratory transpulmonary pressure (EitP) in a method of stepwise increased PEEP level(s), wherein said determination is performed within a number of breathing cycles after an increased PEEP level, and wherein said number of breathing cycles is less than 5.

### RELATED APPLICATIONS

U.S. Provisional Patent Application Ser. No. 61/356,589, European Patent Application EP10166587, filed both filed Jun. 19, 2010, and U.S. Provisional Patent Application Ser. No. 61/469,100 filed Mar. 30, 2011, as well as WO2011/157855A1 all entitled “A system and method for determination of transpulmonary pressure in a patient connected to a breathing apparatus”, as well as PCT/EP2016/061866 published as WO2016/189069A1 and entitled “METHOD, SYSTEM AND SOFTWARE FOR PROTECTIVE VENTILATION”, which all are of same applicant and all are hereby incorporated herein by reference in their entirety for all purposes. The present application claims priority from Swedish patent application number SE2350700-7 filed on Jun. 8, 2023, which also is incorporated herein by reference in its entirety for all purposes.

### BACKGROUND OF THE INVENTION

Patients with acute respiratory failure in need for ventilator treatment in intensive care units show highly varying pathophysiologic conditions of the respiratory system. With regard to the heterogeneity of acute lung injury (ALI) and the more severe acute respiratory distress syndrome (ARDS), the percentage of potentially recruitable lung, i.e. lung tissue that was collapsed but can be opened by a high pressure inflation is up to approximately 60%. One important reason for the heterogeneity is whether the patient has ARDS of pulmonary or extrapulmonary origin, i.e. whether it is the lungs per se or the chest wall and the diaphragm that are mainly affected. In most cases of respiratory failure, both the mechanical conditions of the lung, the stiffness (elastance=E) of the lungs (El) and the stiffness of the containing wall, (Ec), chest wall and diaphragm, play an important role.

During ventilator treatment the mechanical properties of the total respiratory system was hitherto determined by the combined effect of stiffness of the lungs and the stiffness of the chest wall/diaphragm working in series. The lung is a compliant unit within another compliant unit, namely the chest wall and the diaphragm. For optimal ventilator treatment, where risks and benefits of the treatment are balanced, knowledge of the stiffness of the chest wall in relation to the stiffness of the lung is of outmost importance.

For instance, the risk of inducing damage to the sensitive lung tissue by the ventilator treatment is increasing when the lung is very stiff and the chest wall/diaphragm is very soft, where most of the airway pressure generated by the ventilator during inspiration acts solely on the lung, i.e. a high transpulmonary pressure is present. Very little of the pressure applied by the ventilator to the patient is transmitted to the surrounding chest wall and diaphragm.

The apparatus and/or method(s) disclosed in WO2011/157855A1 or WO 2016/189069A1 may be further improved.

The present disclosure is about such advantageous improvement, allowing for improved accuracy and patient safety, amongst other advantages.

### SUMMARY OF THE INVENTION

Accordingly, embodiments of the present invention preferably seek to mitigate, alleviate or eliminate one or more deficiencies, disadvantages or issues in the art, such as the above-identified, singly or in any combination by providing a breathing apparatus, computer program, and method according to the appended patent claims.

Disclosed is a method for determination of End inspiratory transpulmonary pressure (EitP) in a method of stepwise increased PEEP level(s), wherein said determination is performed within a number of breathing cycles after an increased PEEP level, and wherein said number of breathing cycles is less than 5.

Disclosed is also a breathing apparatus having a processing unit configured to determinate an End inspiratory transpulmonary pressure (EitP) in a method of stepwise increased PEEP levels wherein said determination is performed within a number of breathing cycles after an increased PEEP level, and wherein said number of breathing cycles is less than 5.

In examples of said method or apparatus a time of said determination is less than 40 seconds, such as less than 20 seconds.

In examples of said method or apparatus said number of breathing cycles is less than 4, said number of breathing cycles is less than 3, said number of breathing cycles is 2, or said number of breathing cycles is 1.

In examples of said method or apparatus said determination is provided or performed during mechanical ventilation of a patient in volume control mode of a breathing device such as a ventilator or anesthesia machine.

In examples, said breathing device is programmed to deliver a double baseline tidal volume before PEEP is increased during ensuing expiration.

In examples of said method or apparatus said determination is provided or performed during mechanical ventilation of a patient in pressure control mode of a breathing device such as a ventilator or anesthesia machine.

In example, said breathing device is programmed to deliver a tidal volume with the sum of the baseline driving pressure and the increase in PEEP.

In examples of said method or apparatus, said method includes providing a tidal volume with a driving pressure at physiological levels such with a plateau pressure below 45 cm H2O, wherein said driving pressure is more than 1.3 times the driving pressure at baseline PEEP before increasing PEEP to the new level (stepwise increase of PEEP).

In examples of said method or apparatus, said driving pressure is equal to or more than 1.5 times the driving pressure at baseline PEEP before increasing PEEP to the new level (stepwise increase of PEEP).

In examples of said method or apparatus, said driving pressure is equal to or more than 2.0 times the driving pressure at baseline PEEP before increasing PEEP to the new level (stepwise increase of PEEP).

In examples of said method or apparatus, said determination is provided or is performed at regular intervals during mechanical ventilation of a patient, such as once every 30 minutes or once every hour.

In examples of said method or apparatus, said method is a single stepwise increase of said PEEP level, or a multiple stepwise increase of said PEEP levels.

Disclosed is also a method for determination of End inspiratory transpulmonary pressure (EitP) in a method of stepwise increased PEEP levels, such as the method referenced to herein above, wherein said EitP is estimated at a highest PEEP level of said method based on previous PEEP steps, including estimation of lung elastance EL at said highest PEEP level.

Disclosed is also a breathing apparatus, such as the breathing apparatus referenced to herein above, having a processing unit configured to determinate an End inspiratory transpulmonary pressure (EitP) in a method of stepwise increased PEEP levels, wherein said EitP is estimated at a highest PEEP level of said method based on previous PEEP steps, including estimation of lung elastance EL at said highest PEEP level.

In examples of said method or apparatus, an end-inspiratory transpulmonary P/V point at the highest PEEP level is estimated subtracting APPL obtained by linear extrapolation of the APPL of the at least one lower PEEP level from the end-inspiratory airway pressure at the highest PEEP level.

In examples of said method or apparatus, end-inspiratory transpulmonary pressure at the highest PEEP level is estimated by extrapolating a APPL from a APPL at lower PEEP levels

In examples of said method or apparatus, end-inspiratory transpulmonary P/V point at the highest PEEP level is estimated subtracting APPL obtained by linear extrapolation of the APPL of the lower PEEP levels from the end-inspiratory airway pressure at the highest PEEP level.

Disclosed is also a method; an apparatus/device/system; a non-transitory computer-readable storage medium encoded with programming instructions, said storage medium being loaded into a computerized control system said programming instructions causing said computerized control unit to control a breathing device; a breathing apparatus; a computer-readable medium having embodied thereon a computer program for processing by a computer of a breathing apparatus; a computer program enabling carrying out of a method; a graphical user interface; or a use of the apparatus/device/system/software product; in accordance with the enclosed description, claims, abstract and drawings.

### Advantages

The determined EitP is useable as parameter for controlling the breathing apparatus. A clinical decision system may be based on the determined EitP Being able to provide the determined EitP value is hugely advantageous as it is made very patient friendly (no continued high levels of clinically acceptable needed) as well due to its determination speed very computational friendly, cost efficient and clinically acceptable as almost no waiting time is needed. As the determination can be repeated more often (each determination maneuver takes less time) patient care can be improved as e.g. clinically interesting changes in EitP can be determined quickly.

### DRAWINGS

These and other aspects, features and advantages of which embodiments of the invention are capable of will be apparent and elucidated from the following description of embodiments of the present invention, reference being made to the accompanying drawings, in which:

FIGS. 1 and 2 illustrate a measurement procedure;

FIGS. 3 and 4 illustrate another measurement procedure;

FIG. 5 is an illustration is provided of a change in offset between Vti and Vte when PEEP is increased;

FIG. 6 is an illustration is provided of a best-fit linear and 3rd degree polynomial line of cumulative ΔEELV;

FIG. 7 are illustrative CT scans;

FIG. 8 is an illustration of Breath-by-breath build-up of a new PEEP/EELV equilibrium;

FIG. 9 is an illustration of absolute lung volume and Quasistatic pressures;

FIG. 10 is an illustration of a basic PEEP step procedure;

FIG. 11 is a P/V diagram illustrating a PEEP increase procedure;

FIG. 12 is an illustration of an extended two-step PEEP procedure;

FIG. 13 is a P/V diagram of such an extended procedure;

FIG. 14 is a P/V curve;

FIG. 15 are various PTP/V curves;

FIG. 16 is another PTP/V curve illustrating extrapolation of APPL;

FIG. 17 is yet another plot of transpulmonary pressure plotted vs volume;

FIG. 18 illustrates a comparison of two PTP/V curves;

FIG. 19 is a P/V curve;

FIG. 20 are two different PTP/V curves;

FIG. 21 is an illustration of various distributions;

FIG. 22 is yet another PTP/V curve;

FIG. 23 are various PTP/V curves;

FIG. 24 is yet another PTP/V curve;

FIG. 25 is yet another PTP/V curve;

FIG. 26 are two different PTP/V curves;

FIG. 27 is a P/V diagram;

FIG. 28 is a PEEP/Impedance diagram;

FIG. 29 is a PTP/V diagram comparison and overview;

FIG. 30 is a schematic illustration of a breathing apparatus described herein; and

FIG. 31 is a flowchart of a method described herein.

### DESCRIPTION OF THE PREFERRED EMBODIMENTS

Specific embodiments of the invention will now be described with reference to the accompanying drawings. This invention may, however, be embodied in many different forms and should not be construed as limited to the embodiments set forth herein; rather, these embodiments are provided so that this disclosure will be thorough and complete, and will fully convey the scope of the invention to those skilled in the art. The terminology used in the detailed description of the embodiments illustrated in the accompanying drawings is not intended to be limiting of the invention. In the drawings, like numbers refer to like elements.

In “Lung Barometry” measurements as described below were performed in the intensive care of Skane University Hospital in Lund (Sweden). A PEEP-step manoeuvre is completed in less than 20 seconds and a two-PEEP step manoeuvre consequently is completed in less than 40 seconds. As will be seen below, determination of End inspiratory transpulmonary pressure (EitP) is performed within a number of breathing cycles after an increased PEEP level, and wherein said number of breathing cycles is less than 5.

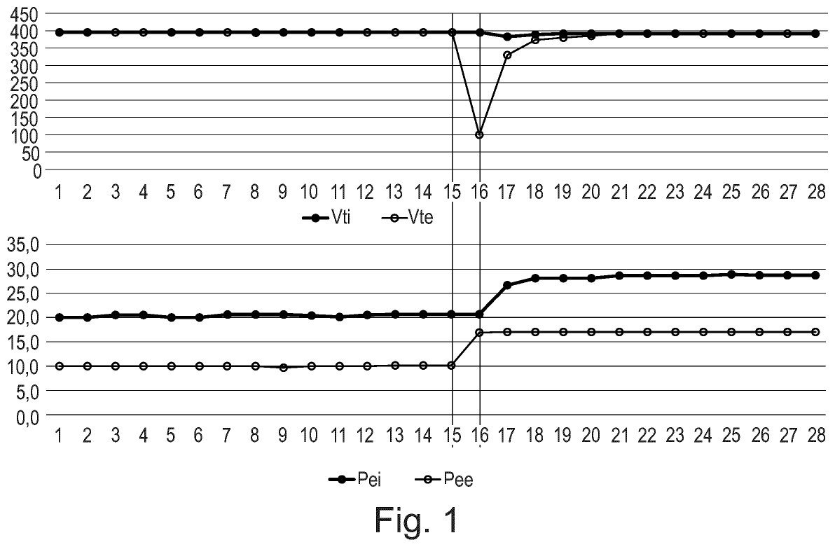

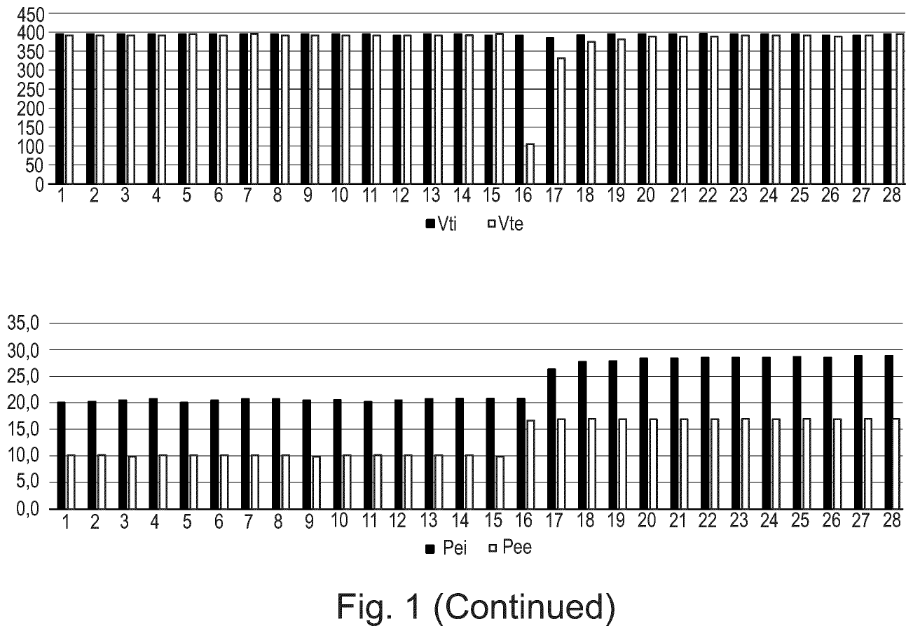

The basis of the measurement procedure is in an example volume control ventilation where the PEEP increase causes a first increase in EELV of around 65% of the tidal volume and a second multi breath phase with a further increase in EELV until the transpulmonary pressure has increased as much as PEEP was increased (see FIG. 1, 2).

FIG. 1 illustrates a One-step measurement procedure in volume control mode. The upper panels and lower panels show inspiratory and expiratory tidal volume (VTi, VTe) and PEEP and end-inspiratory airway plateau pressure (Pee, Pei) as line and bar diagrams. The vertical lines indicate that the last inspiration before PEEP is increased starts from baseline PEEP level and that the first inspiration after the PEEP increase starts from the new PEEP level.

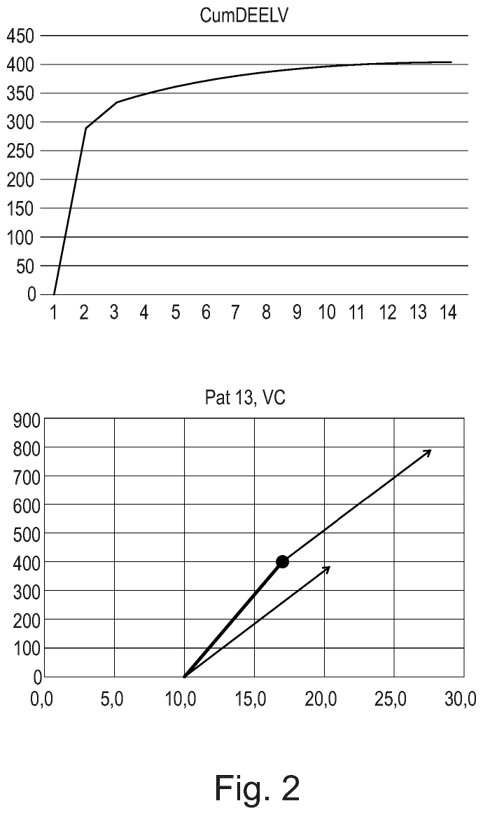

FIG. 2. Left panel: Cumulative ΔEELV (ALSO DENOTED DEELV) breath-by-breath shows that EELV slowly increases and reaches a new plateau level after 14-15 breaths. Right panel: P/V diagram of one-step procedure. Red arrows: tidal airway P/V curves at 10 and 20 cmH2O of PEEP. Blue line: Lung P/V curve between PEEP levels.

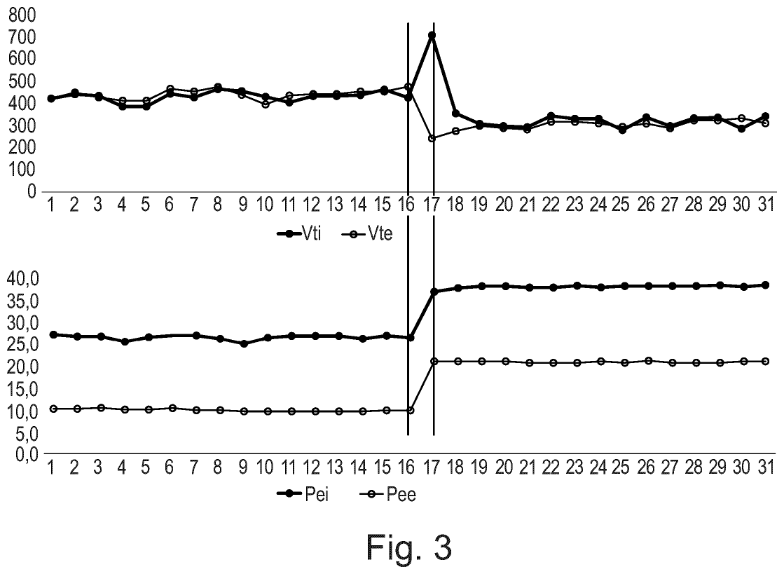

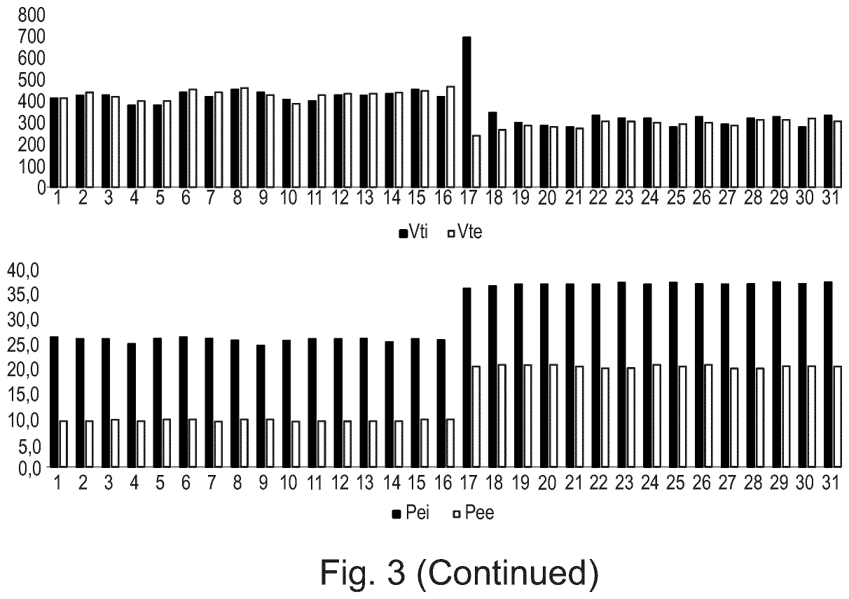

In one of the measurement procedures performed in pressure control ventilation (PC) (patient 9), the driving pressure of the last inspiration before the PEEP increase is the sum of the set driving pressure at baseline and the selected increase in PEEP. This causes a much larger last tidal inspiration followed by an expiration that ends at the selected PEEP level (FIG. 3, 4).

In another example, as shown in FIG. 3, a One-step measurement procedure is preformed and provided in pressure control (or alternatively in pressure support mode). Upper panels and lower panels show inspiratory and expiratory tidal volume (VTi, VTe) and PEEP and end-inspiratory airway plateau pressure (Pee, Pei) as line and bar diagrams. The vertical lines indicate that the driving pressure of the last inspiration before PEEP is the sum of the driving pressure set at baseline, 16 cmH2O and the selected increase in PEEP, 10 cmH2O, a total driving pressure of 26 cmH2O. This results in a tidal volume of around 700 ml, i.e. around 300 ml higher than baseline tidal volumes.

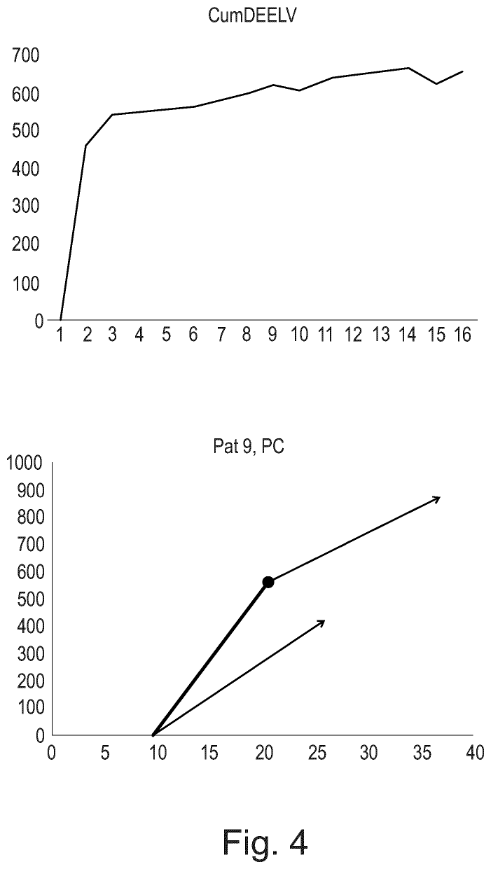

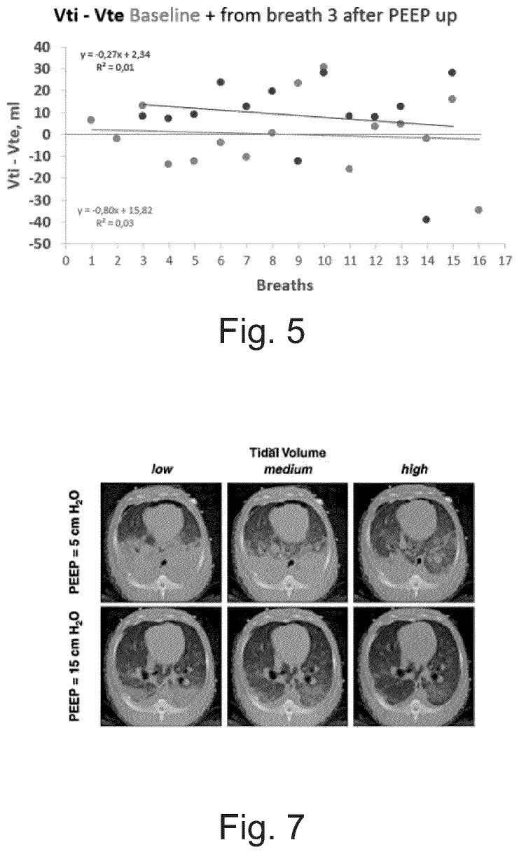

In FIG. 4, the Left panel illustrates Cumulative ΔEELV breath-by-breath shows that the increase in EELV seems to be complete after 2-3 breaths. The almost linear increase in EELV after the 2-3 first breaths is a result of a change in off-set between VTi and VTe when PEEP is increased (FIG. 5). The Right panel in FIG. 4 illustrates in P/V diagram a one-step procedure. The “Red” “arrows” show the tidal airway P/V curves at 10 and 20 cmH2O of PEEP. The “Blue” line shows the Lung P/V curve between PEEP levels.

In FIG. 5 an illustration is provided of a change in offset between Vti and Vte when PEEP is increased analysed as Vti−Vte before and after PEEP up. Breaths number one and two after PEEP up are excluded. Offset during baseline is zero, while mean offset of breaths 3-15 after PEEP up is 9 ml. The almost linear increase in ΔEELV during breaths 3-15 is 112 ml and is a result of change of offset. The true ΔEELV thus is probably the sum of ΔEELV of breaths 1 and 2, 531 ml.

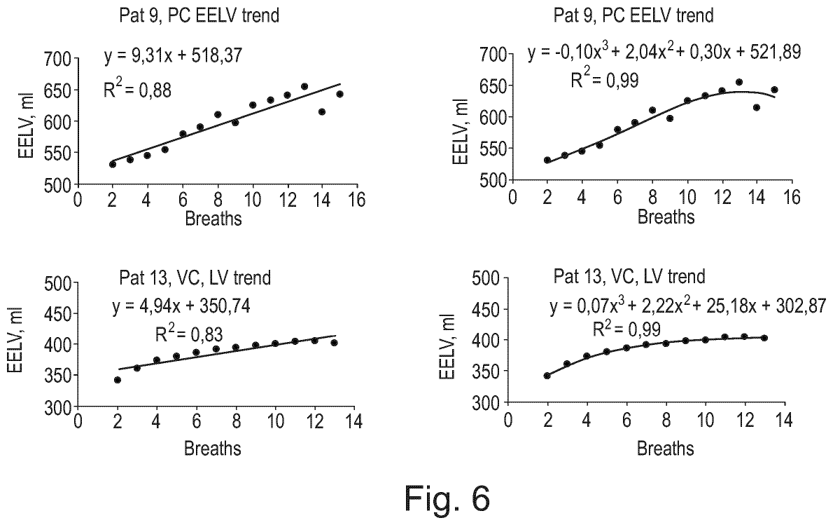

In FIG. 6 an illustration is provided of a best-fit linear and 3rd degree polynomial line of cumulative ΔEELV from the second breath after increasing PEEP and onwards in PC and VC. This supports the notion that the full ΔEELV is reached in two breaths in pressure control/support ventilation due to the large pre-PEEP up tidal volume (FIG. 6).

In FIG. 7 a representative CT scan is shown that is obtained in one dog at end-expiration for each experimental step. A similar PEEP level, either 5 or 15 cm H2O, the amount of end-expiratory collapse was dramatically different, depending on whether ventilation was performed at low, medium, or high VT. Best linear fit lines for cumulative ΔEELV from second breath and onwards in PC (upper left panel) and VC (lower left panel) show r2=0.88 in PC and 0.83 in VC. In contrast a 3rd degree polynomial best-fit curve in VC has an r2 of 0.99 while in PC, such a best-fit curve show a non-plausible form and only reaches a r2 of 0.94. This further supports that the increase of EELV after the two first breaths in PC is a result of a change in offset between VTi and VTe after increasing PEEP.

From a lung mechanical point of view, the effect of the large pre-PEEP inspiration seems plausible and logical, as the expiration after that starts from a very high volume. Expiration is passive and caused by the recoil of the lung, while the expansive chest wall counter-acts expiration. The expiration stops when airway pressure reaches the new PEEP level (20 cmH2O) and at this volume level, the chest wall is off-loaded from the lung by the chest wall spring out force. In a study by the Gattinoni group on the effect of intermittent sighs during mechanical ventilation (Pelosi P, Cadringher P, Bottino N, Panigada M, Carrieri F, Riva E, Lissoni A, Gattinoni L: Sigh in acute respiratory distress syndrome. American journal of respiratory and critical care medicine 1999, 159(3):872-880) it was shown that EELV increased by around 400 ml when sighs were implemented without increasing PEEP. In another study by the Gattinoni group that large tidal volumes, as compared to smaller tidal volumes, increased EELV without increase in PEEP and it did not matter if the tidal volumes started from 5 or 20 cmH2O of PEEP (Pelosi P, Goldner M, McKibben A, Adams A, Eccher G, Caironi P, Losappio S, Gattinoni L, Marini J J: Recruitment and derecruitment during acute respiratory failure: an experimental study. American journal of respiratory and critical care medicine 2001, 164(1):122-130).

This indicates that a sudden increase in tidal volume pushes the chest wall to a new higher volume and ensuing expiration ends at a higher EELV than before the sigh. In the present recording in pressure control mode, where PEEP is increased to 20 cmH2O during the first expiration after the large inspiration of 691 ml, expiratory tidal volume is only 237 ml. This means that the first expiration ΔEELV is 454 ml, which is 30 ml higher than the normal baseline tidal volumes. In volume control ventilation, the first expiration ΔEELV is on average only 65% of the tidal volume at baseline.

The reason why it takes 2-3 breaths to complete the build-up of ΔEELV is the fact that airway resistance and the short time for expiration in relation to the large tidal volume prevent a single expiration completion of ΔEELV.

Findings suggest that a one-step measurement procedure is performed in 4-5 breaths (less than seconds) and that it is in examples performed in both pressure control, pressure support mode or volume control mode.

In volume control mode, the ventilator is preferably configured and/or programmed to deliver a double baseline tidal volume before PEEP is increased during the ensuing expiration.

In pressure control ventilation, the ventilator preferably configured and/or programmed programmed to deliver a tidal volume with the sum of the baseline driving pressure and the increase in PEEP.

In both pressure and volume based ventilation modes, the time for expiration after the large inspiration is preferably at least doubled to allow for a completion of ΔEELV build up in as few breaths as possible.

The decrease in duration of the measurement procedure makes the effect of high PEEP on hemodynamics minimal.

The driving pressure is for instance equal to or more than 1.5 times the driving pressure at baseline PEEP before increasing PEEP to the new level (stepwise increase of PEEP).

In some examples, said driving pressure is equal to or more than 2.0 times the driving pressure at baseline PEEP before increasing PEEP to the new level (stepwise increase of PEEP).

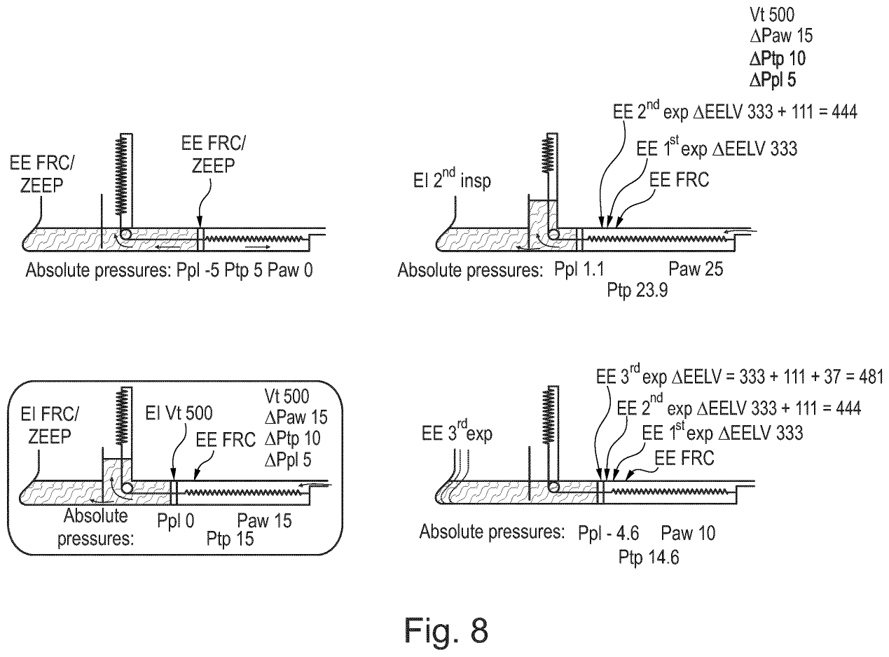

In FIG. 8 a Breath-by-breath build-up of a new PEEP/EELV equilibrium is illustrated in model. Note, that transpulmonary pressure at end-inspiration of a tidal volume of 500 ml (in red square) is equal to transpulmonary pressure at end-expiration at a PEEP of 10 cmH2O and a ΔEELV of 500 ml, i.e. an increase in EELV of 500 ml above FRC (the figures in a “red” square).

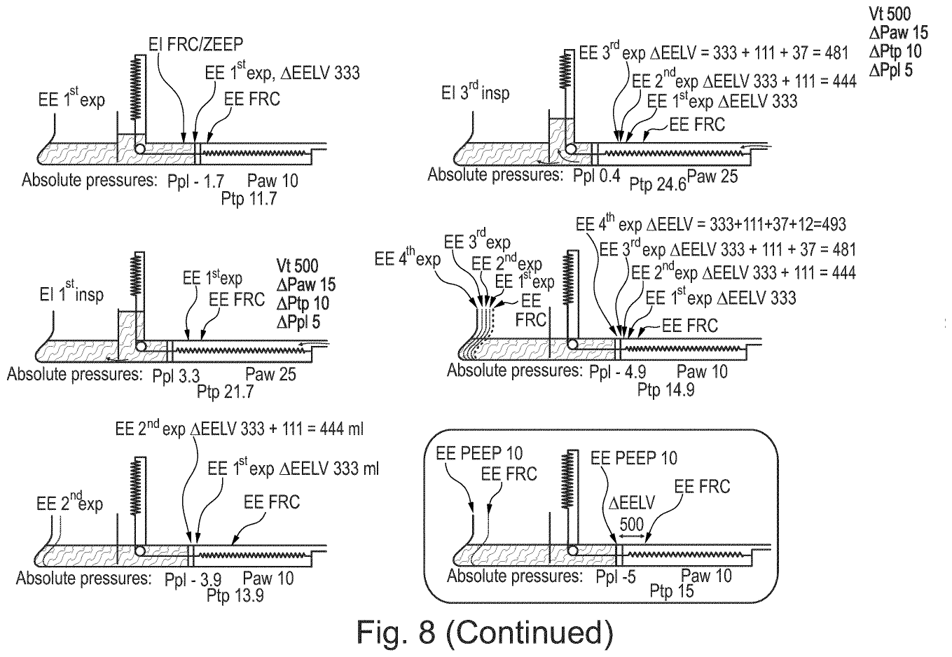

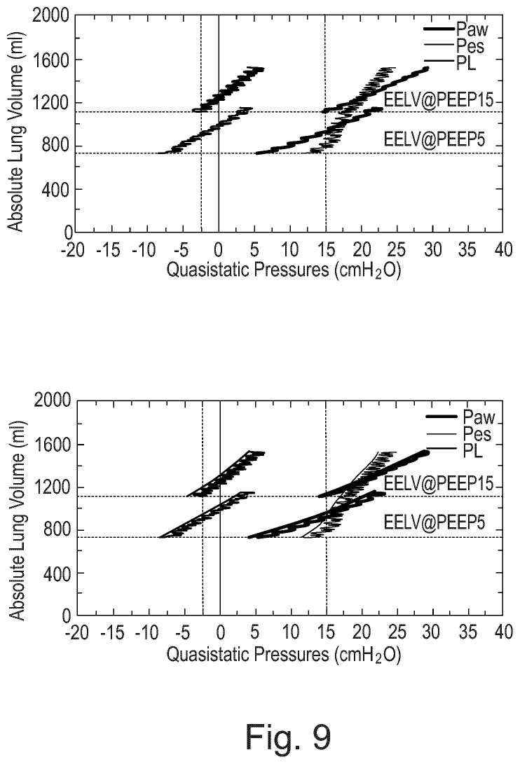

FIG. 9 is an illustration of absolute lung volume and Quasistatic pressures. The Upper panel in FIG. 9 illustrates an Original registration (L. Chen of Brochard group, Toronto) of airway, and esophageal pressure at 5 and 15 cmH2O of PEEP of patient in Chen et al. Am J Resp Crit Care Med 2020; 201(2):178-87 (1). The lower panel of FIG. 9 shows whole lines inserted to eliminate cardiac pressure variations to enhance analysis.

With reference to FIG. 31, a method 350 is disclosed including determining 365 of End inspiratory transpulmonary pressure (EitP) in a method of stepwise increased PEEP level(s). The determination is performed within a number of breathing cycles 370 after increasing a PEEP level 360. The number of breathing cycles is less than 5.

With reference to FIG. 30 a breathing apparatus 300 is disclosed having a processing unit 310 configured to determinate an End inspiratory transpulmonary pressure (EitP) in a method of stepwise increased PEEP levels. The processing unit is configured to execute a software for this. The software is performing the method disclosed in the previous paragraph and elsewhere herein. The software is stored on a computer readable medium of/accessible by the breathing apparatus 300 or other suitable hardware, e.g. e personal digital communication device. Determination may in examples be made remote from the breathing apparatus, but based on input from sensors of the breathing apparatus known in the art. The determination is performed within a number of breathing cycles after an increased PEEP level, and said number of breathing cycles is less than 5.

### Calculation of the Lung P/V Curve

The calculation of a lung P/V curve during a PEEP trial is preferably performed using end-expiratory and end-inspiratory airway pressure and measurements of ΔEELV by the cumulative expiratory tidal volume method, i.e. without using esophageal pressure data. Besides being simpler, this improves precision of measurements, as precision of esophageal pressure measurements is hampered by their dependency on the calibration procedure, the Baydur maneuver, as esophageal pressure is not representative of mean pleural pressure. The Baydur maneuver is regarded as acceptable if the APES during the maneuver is within a range of 0.8-1.2 of the ΔPAW caused by the compression of the thorax, which means that APES precision is very low.

### Determining ΔEELV

For the highest precision of ΔEELV measurement, ΔEELV is preferably determined directly and not indirectly as the difference of EELV measurements by nitrogen washin/washout (N2 Wi/Wo) between two PEEP levels. The N2 Wi/Wo method has typically a variability in measurements of ±10%. In a case with an EELV of 1800 ml at the low PEEP and 2100 ml at the high PEEP level, i.e. a ΔEELV of 300 ml, the EELV at the low PEEP can be between 1620 and 1980 ml and EELV at the high PEEP level can be between 1890 and 2310 ml. As a consequence, ΔEELV measured by washin/washout can be between 690 ml and −90 ml, which is an unacceptable span.

In contrast, determination of ΔEELV by the cumulative expiratory tidal volume difference method is very precise as the inspiratory tidal volume varies less than 1% and can be regarded as constant during a PEEP step maneuver. Thus, in fact the method determines ΔEELV directly as the cumulative difference in only expiratory tidal volume. We have made repeated PEEP steps in ARDS patients and shown that in its revised version the precision of ΔEELV measurement is ±3%, which in the above described case would mean that a ΔEELV of 300 ml would be between 291 and 309 ml. This method for direct measurement of ΔEELV is not just an estimation, it is a very precise measurement method.

### Basic One-Step PEEP Procedure

PEEP is increased from baseline clinical level 6-10 cmH2O and then back again. ΔEELV up and down is determined and the procedure is finalized by setting the tidal volume to the mean of ΔEELV up and down.

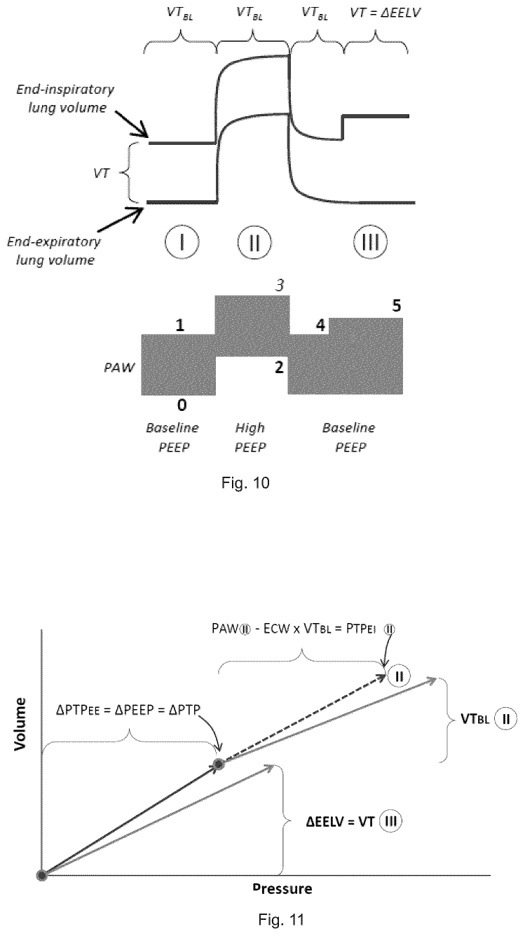

In FIG. 10 a basic PEEP step procedure is illustrated. Roman digits are referring to the three different parts of the measurement procedure. The arabic digits are identifying end-inspiratory and end-expiratory P/V points where transpulmonary pressure can be determined. Italics indicate where transpulmonary pressure only is estimated.

In FIG. 11 a P/V diagram is provided illustrating of the PEEP increase basic procedure. “Red” arrows: tidal airway P/V curves. “Blue” arrows: tidal transpulmonary P/V curves. Dashed “blue” line: estimated curve based on the assumption that chest wall elastance is almost constant when increasing PEEP.

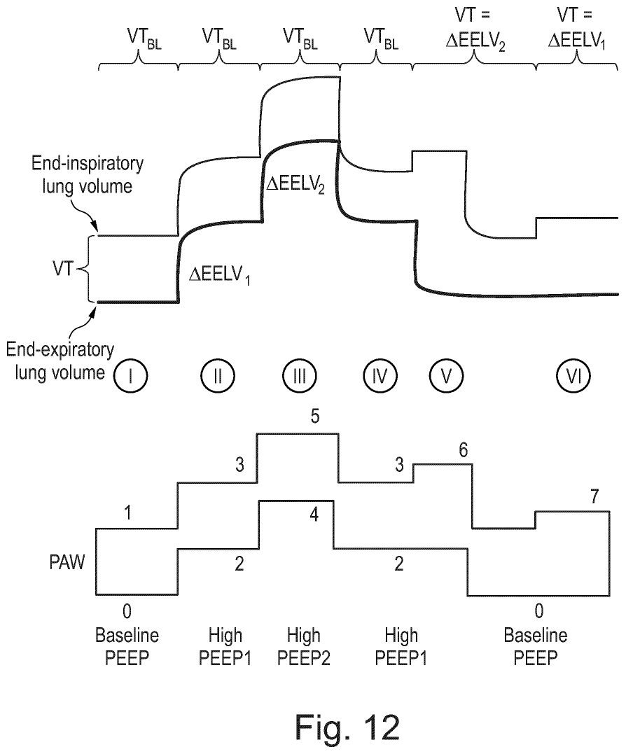

Extended two-step PEEP procedure See FIG. 12. PEEP is increased in two steps from baseline level and then lowered again from the highest PEEP level to the first PEEP level above baseline. ΔEELV up and down for the highest PEEP step are determined and the tidal volume is set equal to mean ΔEELV 2. Then PEEP is lowered to baseline PEEP level and ΔEELV up and down for the first PEEP step is determined and the procedure finalized by setting the tidal volume equal to mean ΔEELV 1.

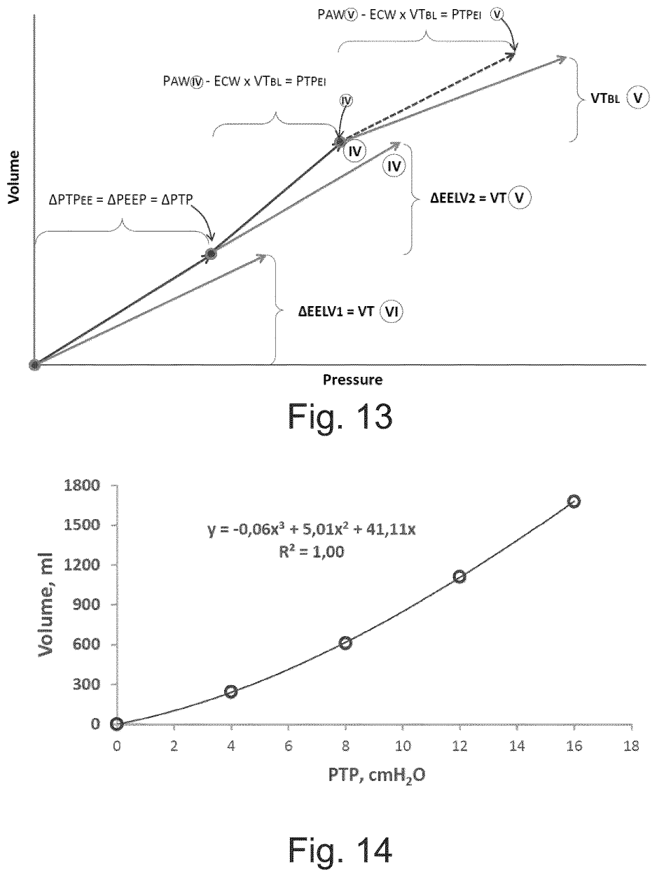

FIG. 13 P/V diagram of an extended procedure. “Red” lower arrows”: tidal airway P/V curves. “Blue” upper arrows: tidal transpulmonary P/V curves. Dashed “blue” arrow/line: estimated curve based on the assumption that chest wall elastance is almost constant when increasing PEEP.

### Calculation of Full Lung P/V Curve

Step 1: The lung P/V curve is obtained by plotting the end-expiratory transpulmonary pressure (PAW_{EE}) versus the end-expiratory lung volume at each PEEP level (see FIG. 14). When lung elastance is determined as the difference in end-expiratory airway pressure divided by the difference in volume between two PEEP levels, this is the true lung elastance, as at end-expiration at a steady state PEEP/EELV equilibrium, the static airway pressure measured is equal to static transpulmonary pressure (see FIG. 6). This means that lung elastance is determined directly in contrast to the conventional method, where lung elastance is determined indirectly as the difference between respiratory system elastance and chest wall elastance. The direct method for determining lung elastance (ΔPAW_{EE}/ΔEELV) is extremely precise, as the end-expiratory airway pressure is maintained by the ventilator within mmH2O of set value and reflects the mean transpulmonary pressure of the whole lung.

It can be seen from e.g. FIG. 14 that as the end-expiratory transpulmonary pressure increases as much as PEEP is increased and the increase in end-expiratory lung volume following a PEEP increase is equal to DPEEP/EL, a lung P/V curve can be obtained by plotting the end-expiratory airway pressure vs cumulative end-expiratory lung volume.

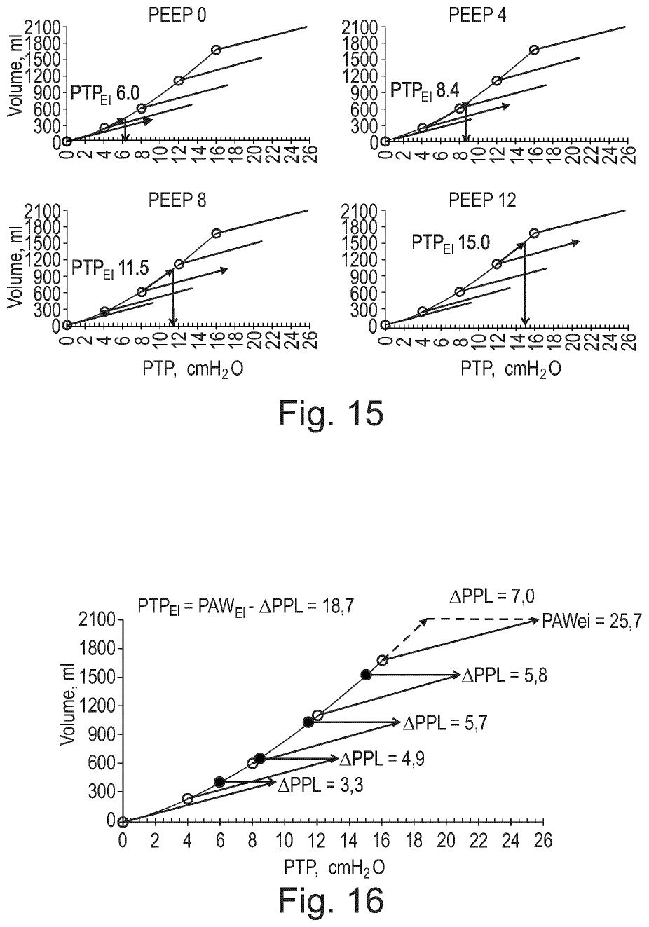

Step 2: As the end-expiratory and end-inspiratory lung P/V curves coincide and forms a common single lung P/V curve, the end-inspiratory transpulmonary pressure at each PEEP level can be determined by solving the best-fit equation for the lung P/V curve for the end-inspiratory lung volume at each PEEP level (see FIG. 15).

It can be seen from e.g. FIG. 15 that the end-inspiratory transpulmonary pressure, PTPei, is determined by solving the equation for the lung P/V curve at the end-expiratory lung volume at each PEEP level. Blue arrows: tidal lung P/V curves. Black arrows point at the end-inspiratory pressure level.

Step 3: The end-inspiratory transpulmonary pressure at the highest PEEP level (16 cmH2O) cannot be determined exactly as the lung P/V curve is non-linear and the chest wall elastance increases slightly PEEP step by PEEP step. Instead, the end-inspiratory transpulmonary pressure at the highest PEEP level can be estimated by extrapolating the DPPL (APPI, change in pleural pressure) from the DPPL at the four lower PEEP levels (see FIG. 16).

It can be seen from e.g. FIG. 16 that the end-inspiratory transpulmonary P/V point at the highest PEEP level is estimated subtracting DPPL obtained by linear extrapolation of the DPPL of the four lower PEEP levels from the end-inspiratory airway pressure at the highest PEEP level. In this case shown in FIG. 16, the extrapolated APPL is 7.0 cmH2O and as the PAWEI is 25.7 cmH2O, the PTPEI is 18.7 cmH2O.

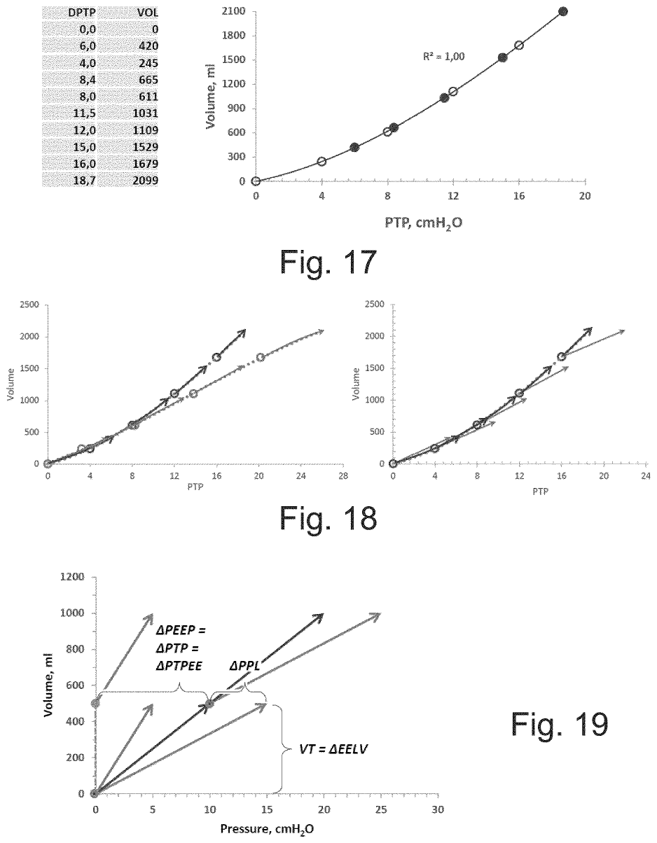

By the calculation procedures described above, the static steady state end-expiratory and end-inspiratory pressure and volume points are obtained for the whole PEEP trial from end-expiration at the lowest PEEP level to end-inspiration of the highest PEEP level (see FIG. 17). This is illustrated in an example in FIG. 17, where transpulmonary pressure plotted vs volume during PEEP steps 0-4-8-12-16 cmH2O. Open circles: End-expiratory P/V points. Filled circles: End-inspiratory P/V points. Note that end-expiratory and end-inspiratory p/V points are aligned on a single common lung P/V curve.

It should be noted that there is only one solution for calculation of the lung P/V curve where the tidal lung elastance, the end-expiratory lung elastance and the end-expiratory airway (respiratory system) elastance are equal and the end-expiratory and end-inspiratory transpulmonary pressure at a certain lung volume is the same, independent of whether this volume has been reached by tidal inflation or PEEP inflation. This can be illustrated by comparing the end-expiratory airway P/V curve (the lung P/V curve) with a lung P/V curve, where EL is determined conventionally as (ΔPAW−ΔPES)/VT (see FIG. 18).

With reference to FIG. 18, the lung P/V curve calculated as described in FIGS. 15-18, where the expiratory lung curve is indicated by a red dotted line and the tidal lung P/V curves indicated by dark blue arrows.

The left panel in FIG. 18 shows a Comparison with lung P/V curve where the increase in end-expiratory transpulmonary pressure is calculated as ΔEELV×EL, where EL is determined conventionally as (ΔPAW−ΔPES)/VT. Note that the tidal lung P/V curves (grey arrows) are aligned on a single lung P/V curve gray dotted line), but the end-expiratory transpulmonary P/V points are not coinciding with the end-expiratory airway P/V points, and the whole lung P/V curve, as a consequence, right shifted in relation to the end-expiratory airway P/V curve.

The right panel in FIG. 18 shows a comparison where tidal lung P/V curves start from end-expiratory airway P/V points, where the tidal lung P/V curves are right shifted from the end-expiratory airway P/V curve. Consequently, tidal inspiratory lung P/V curves do not coincide with the end-expiratory airway P/V curve.

### Calibration of Esophageal Balloon Positioning and Filling

The implication of the coinciding end-expiratory airway, end-expiratory transpulmonary and tidal inspiratory P/V curves are, that when the tidal volume is equal to ΔEELV, the tidal pleural pressure variations are equal to the difference between the end-inspiratory airway pressure from the low PEEP and the end-expiratory airway pressure of the high PEEP (see FIG. 19). For somebody who wants to use esophageal pressure, the correct filling of the balloon will be achieved when the tidal esophageal pressure changes are equal to PAW_{EI}_{_{LOPEEP}}−PAW_{EE}_{_{HIPEEP}}, as this is a true reference pleural tidal pressure swing.

With reference to FIG. 19, Tidal P/V curves are shown including: airway: “red” (lowest) arrows, lung (transpulmonary): “blue” (mid) arrows, chest wall (pleura): “green” (upper) arrows. Dashed “green” line: end-expiratory chest wall P/V curve (ΔPPLEE/ΔEELV). PEEP has been increased so ΔEELV has increased to a level equal to the tidal volume at baseline PEEP (500 ml), as ΔEELV is determined by the size of the PEEP step (10 cmH2O) and lung elastance (20 cmH2O/L). End-expiratory transpulmonary pressure increase, calculated as ΔEELV×EL, is 10 cmH2O, and this means that end-expiratory transpulmonary pressure at the high PEEP level is equal to the end-inspiratory transpulmonary pressure of the tidal volume at baseline PEEP, which means that the transpulmonary driving pressure of a tidal volume equal to ΔEELV, is equal to ΔPEEP. Consequently, transpulmonary pressure at a certain lung volume level is independent of mode of inflation: tidal or PEEP inflation and the tidal pleural pressure changes are equal to PAW_{EI}_{_{LOPEEP}}−PAW_{EE}_{_{HIPEEP}}.

### Regional Lung P/V Curves

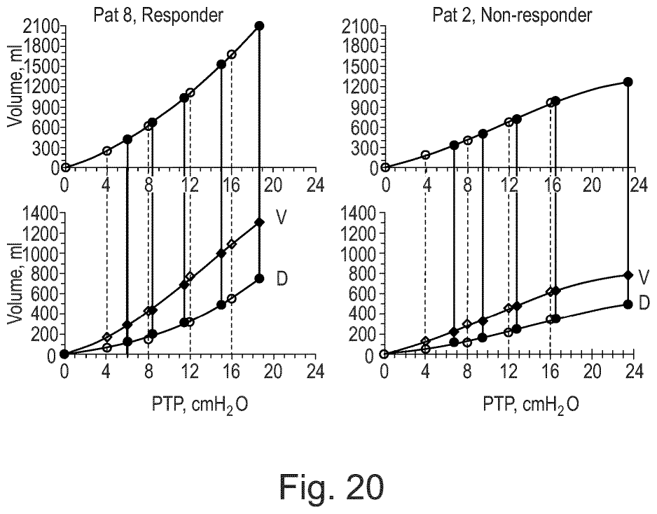

As all pressures are measured/derived during steady state, static conditions, these pressures are the same in the whole lung from non-dependent to dependent regions. Consequently, gas will be distributed according to the regional elastic properties of the lung, both the tidal volume and ΔEELV. This is confirmed by using the EIT signal to divide the volume changes in the upper and lower half of the lung, which shows that end-expiratory and end-inspiratory transpulmonary P/V points are aligned on a common single lung P/V curve in both the non-dependent (ventral) and dependent (dorsal) half of the lung (see FIG. 20).

The lung P/V curve passes through the end-expiratory airway P/V points and show a decreasing lung elastance when PEEP is increased in the responder and an increasing lung elastance in the non-responder (see FIG. 20).

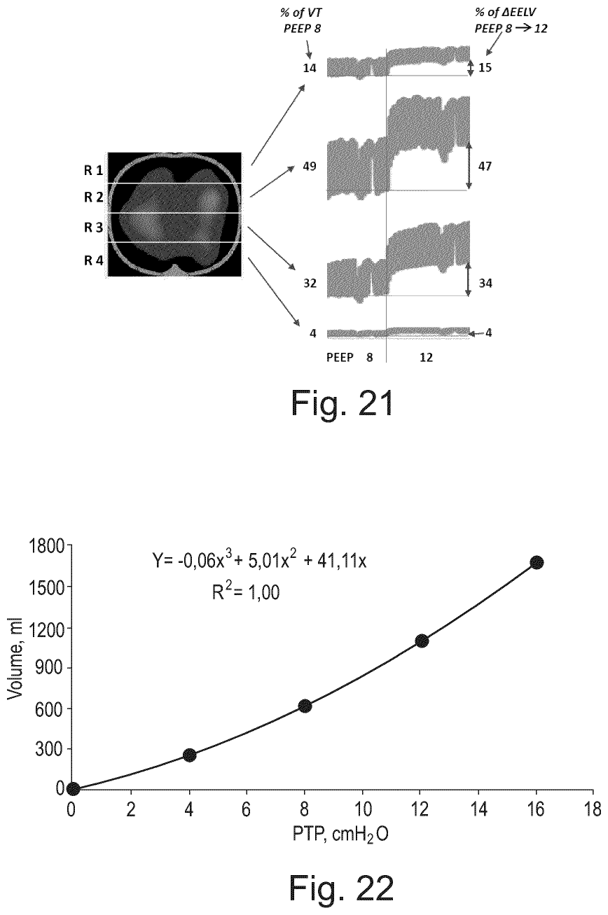

With reference to FIG. 20, lung P/V curves are illustrated for a PEEP responder (left panel) and PEEP non-responder (right panel). Upper panels: Whole lung. Lower panels: Lung P/V curves in ventral and dorsal lung. End-expiratory (open circles) and end-inspiratory (closed circles) transpulmonary P/V points. Dashed vertical lines connect end-expiratory P/V points and whole vertical lines connect end-inspiratory transpulmonary P/V points in the whole lung and ventral and dorsal lung separately, as static pressures are the same everywhere in the lung. Also, the distribution of a tidal volume from a low PEEP level is equal to the distribution of ΔEELV between the low and the high PEEP level, when the tidal volume is equal to the change in end-expiratory lung volume, VT=ΔEELV (see FIG. 21).

With reference to FIG. 21, a distribution is illustrated in four equally high regions from the most non-dependent to the most dependent region of a tidal volume from PEEP 8 cmH2O and the end-expiratory lung volume change between PEEP 8 and 12 cmH2O, in a case where VT=ΔEELV and there is a PEEP/ΔEELV equilibrium at each PEEP level.

Lung and Chest Wall Mechanics During PEEP Inflation. II.
Calculation of Full Lung P/V Curve from PEEP Trial

Step 1: The lung P/V curve is obtained by plotting the end-expiratory transpulmonary pressure (PAW_{EE}) versus the end-expiratory lung volume at each PEEP level (see FIG. 22). When lung elastance is determined as the difference in end-expiratory airway pressure divided by the difference in volume between two PEEP levels, this is the true lung elastance, as at end-expiration at a steady state PEEP/EELV equilibrium, the static airway pressure measured is equal to static transpulmonary pressure (see FIG. 22). This means that lung elastance is determined directly in contrast to the conventional method, where lung elastance is determined indirectly as the difference between respiratory system elastance and chest wall elastance. The direct method for determining lung elastance (ΔPAW_{EE}/ΔEELV) is extremely precise, as the end-expiratory airway pressure is maintained by the ventilator within mmH₂O of set value and reflects the mean transpulmonary pressure of the whole lung.

With reference to FIG. 22: As the end-expiratory transpulmonary pressure increases as much as PEEP is increased and the increase in end-expiratory lung volume following a PEEP increase is equal to ΔPEEP/EL, a lung P/V curve can be obtained by plotting the end-expiratory airway pressure vs cumulative end-expiratory lung volume.

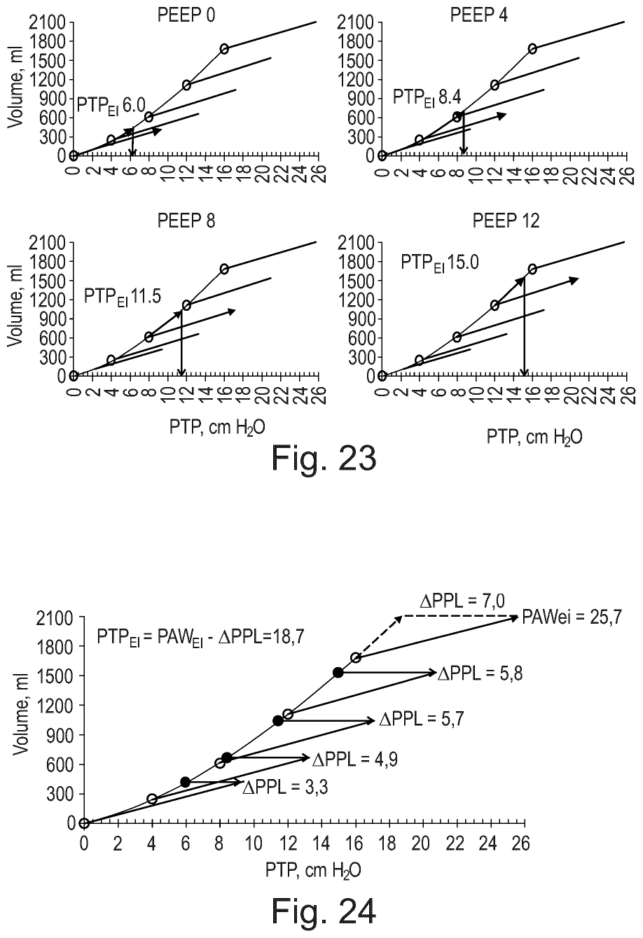

Step 2: As the end-expiratory and end-inspiratory lung P/V curves coincide and forms a common single lung P/V curve, the end-inspiratory transpulmonary pressure at each PEEP level can be determined by solving the best-fit equation for the lung P/V curve for the end-inspiratory lung volume at each PEEP level (see FIG. 23).

With reference to FIG. 23: The end-inspiratory transpulmonary pressure, PTPEI, is determined by solving the equation for the lung P/V curve at the end-expiratory lung volume at each PEEP level. Blue arrows: tidal lung P/V curves. Black arrows point at the end-inspiratory pressure level.

Step 3: The end-inspiratory transpulmonary pressure at the highest PEEP level (16 cmH₂O) cannot be determined exactly as the lung P/V curve is non-linear and the chest wall elastance increases slightly PEEP step by PEEP step. Instead, the end-inspiratory transpulmonary pressure at the highest PEEP level can be estimated by extrapolating the APPL from the APPL at the four lower PEEP levels (see FIG. 24).

With reference to FIG. 24: The end-inspiratory transpulmonary P/V point at the highest PEEP level is estimated subtracting APPL obtained by linear extrapolation of the APPL of the four lower PEEP levels from the end-inspiratory airway pressure at the highest PEEP level. In this case, the extrapolated APPL is 7.0 cmH₂O and as the PAW_{EI} is 25.7 cmH₂O, the PTP_{EI} is 18.7 cmH₂O.

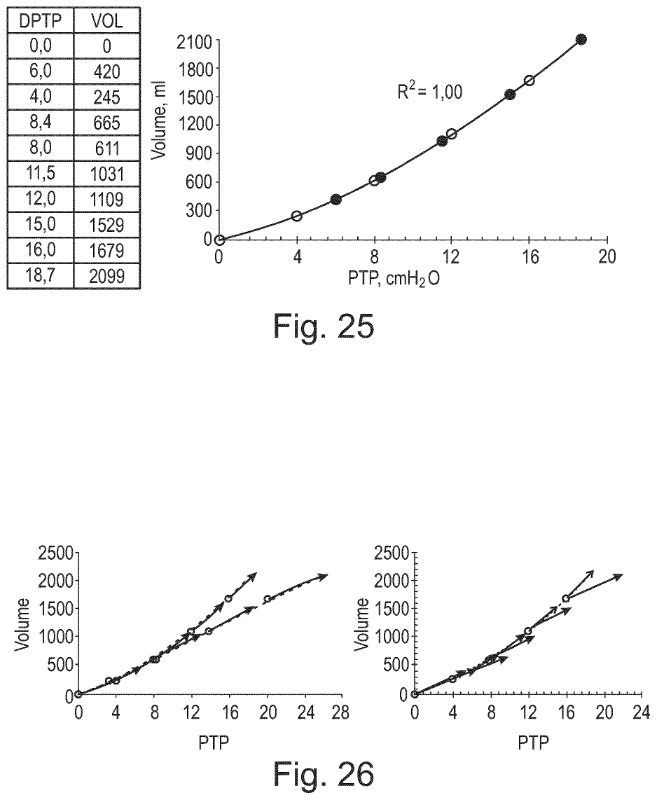

By the calculation procedures described above, the static steady state end-expiratory and end-inspiratory pressure and volume points are obtained for the whole PEEP trial from end-expiration at the lowest PEEP level to end-inspiration of the highest PEEP level (see FIG. 25).

With reference to FIG. 25. Transpulmonary pressure plotted vs volume during PEEP steps 0-4-8-12-16 cmH₂O. Open circles: End-expiratory P/V points. Filled circles: End-inspiratory P/V points. Note that end-expiratory and end-inspiratory p/V points are aligned on a single common lung P/V curve.

There is only one solution for calculation of the lung P/V curve where the tidal lung elastance, the end-expiratory lung elastance and the end-expiratory airway (respiratory system) elastance are equal and the end-expiratory and end-inspiratory transpulmonary pressure at a certain lung volume is the same, independent of whether this volume has been reached by tidal inflation or PEEP inflation. This can be illustrated by comparing the end-expiratory airway P/V curve (the lung P/V curve) with a lung P/V curve, where EL is determined conventionally as (ΔPAW−ΔPES)/VT (see FIG. 26).

With reference to FIG. 26: The lung P/V curve calculated as described in FIGS. 5-8, where the expiratory lung curve is indicated by a red dotted line and the tidal lung P/V curves indicated by dark blue arrows.

With reference to FIG. 26 Left panel: Comparison with lung P/V curve where the increase in end-expiratory transpulmonary pressure is calculated as ΔEELV×EL, where EL is determined conventionally as (ΔPAW−ΔPES)/VT. Note that the tidal lung P/V curves (grey arrows) are aligned on a single lung P/V curve gray dotted line), but the end-expiratory transpulmonary P/V points are not coinciding with the end-expiratory airway P/V points, and the whole lung P/V curve, as a consequence, right shifted in relation to the end-expiratory airway P/V curve.

With reference to FIG. 26 Right panel: Comparison where tidal lung P/V curves start from end-expiratory airway P/V points, where the tidal lung P/V curves are right shifted from the end-expiratory airway P/V curve. Consequently, tidal inspiratory lung P/V curves do not coincide with the end-expiratory airway P/V curve.

### Calibration of Esophageal Balloon Positioning and Filling

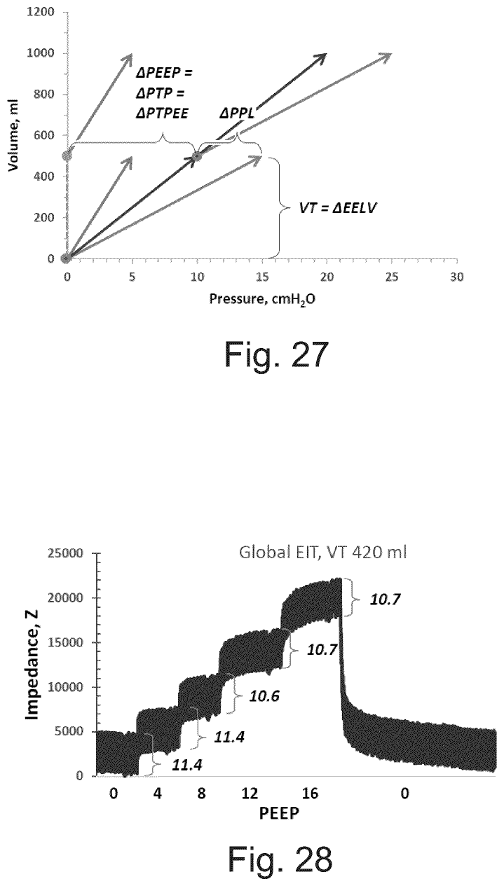

The implication of the coinciding end-expiratory airway, end-expiratory transpulmonary and tidal inspiratory P/V curves are, that when the tidal volume is equal to ΔEELV, the tidal pleural pressure variations are equal to the difference between the end-inspiratory airway pressure from the low PEEP and the end-expiratory airway pressure of the high PEEP (see FIG. 27). For somebody who wants to use esophageal pressure, the correct filling of the balloon will be achieved when the tidal esophageal pressure changes are equal to PAWei_{LOPEEP}−PAWEE_{HIPEEP}, as this is a true reference pleural tidal pressure swing.

With reference to FIG. 27: Tidal P/V curves: airway: red arrows, lung (transpulmonary): blue arrows, chest wall (pleura): green arrows. Dashed green line: end-expiratory chest wall P/V curve (ΔPPLEE/ΔEELV).

PEEP has been increased so ΔEELV has increased to a level equal to the tidal volume at baseline PEEP (500 ml), as ΔEELV is determined by the size of the PEEP step (10 cmH₂O) and lung elastance (20 cmH₂O/L). End-expiratory transpulmonary pressure increase, calculated as ΔEELV×EL, is 10 cmH₂O, and this means that end-expiratory transpulmonary pressure at the high PEEP level is equal to the end-inspiratory transpulmonary pressure of the tidal volume at baseline PEEP, which means that the transpulmonary driving pressure of a tidal volume equal to ΔEELV, is equal to ΔPEEP. Consequently, transpulmonary pressure at a certain lung volume level is independent of mode of inflation: tidal or PEEP inflation and the tidal pleural pressure changes are equal to PAW_{EI}_{_{LOPEEP}}−PAW_{EE}_{_{HIPEEP}}.

### Regional Lung P/V Curves

As all pressures are measured/derived during steady state, static conditions, these pressures are the same in the whole lung from non-dependent to dependent regions. Consequently, gas will be distributed according to the regional elastic properties of the lung, both the tidal volume and ΔEELV. This is confirmed by using the EIT signal to divide the volume changes in the upper and lower half of the lung, which shows that end-expiratory and end-inspiratory transpulmonary P/V points are aligned on a common single lung P/V curve in both the non-dependent (ventral) and dependent (dorsal) half of the lung (see FIG. 28).

### Calculation of Regional Tidal Volume and End-Expiratory Lung Volume Changes

First, the ratio of tidal impedance variation (ΔZ) to tidal volume (ml) is determined for each PEEP level. The tidal volume is constant, 420 ml during the whole PEEP trial.

With reference to FIG. 28: Global EIT registration in PEEP responder. The digits after the red brackets show the ΔZ/ml.

The regional impedance change (ΔZ) related to tidal ventilation is registered from the regional EIT curves. The percentage ΔZ of each region at each PEEP level is calculated. The regional tidal volume for each PEEP level is calculated as percent ΔZ times tidal volume (ml) (Table 1).

TABLE 1
Example of determining regional tidal ventilation.

| | | ΔZ/VT | | | | | | | | | | | | |
|---|---|---|---|---|---|---|---|---|---|---|---|---|---|---|
| | | | | | | Σ ΔZ | %ΔZ | | | | VT ml | | | |
| VT | PEEP | R1 | R2 | R3 | R4 | R1-4 | R1 | R2 | R3 | R4 | R1 | R2 | R3 | R4 |
| 420 | 0 | 720 | 2630 | 1010 | 440 | 4800 | 0.15 | 0.55 | 0.21 | 0.09 | 63 | 230 | 88 | 39 |
| 420 | 4 | 550 | 2590 | 1130 | 530 | 4800 | 0.11 | 0.54 | 0.24 | 0.11 | 48 | 227 | 99 | 46 |
| 420 | 8 | 310 | 2450 | 1080 | 630 | 4470 | 0.07 | 0.55 | 0.24 | 0.14 | 29 | 230 | 101 | 59 |
| 420 | 12 | 390 | 2220 | 1160 | 730 | 4500 | 0.09 | 0.49 | 0.26 | 0.16 | 36 | 207 | 108 | 68 |
| 420 | 16 | 255 | 2000 | 1180 | 1000 | 4435 | 0.06 | 0.45 | 0.27 | 0.23 | 24 | 189 | 112 | 95 |

PEEP Responder patient. R = region of interest. R1: ventral ROI, R2: Mid ventral ROI, R3: mid dorsal ROI, R4: dorsal ROI. VT = tidal volume. ΔZ = tidal impepedance variation.

The regional change in end-expiratory lung volume was calculated as the regional ΔZ/ΔEELV divided by ΔZ/ml determined as the tidal impedance change divided by the tidal volume (Table 2).

TABLE 2
regional ΔEELV linearized and converted to volume (ml) for each
PEEP step by dividing ΔZ/ΔEELV with ΔZ/ml.

| | | ΔZ/ΔEELV | | | | ΔEELV ml | | | |
|---|---|---|---|---|---|---|---|---|---|
| PEEP | ΔZ/ml | R1 | R2 | R3 | R4 | R1 | R2 | R3 | R4 |
| 4 | 11.4 | 450 | 1670 | 430 | 230 | 39 | 146 | 38 | 20 |
| 8 | 11.4 | 600 | 2390 | 680 | 400 | 53 | 209 | 60 | 35 |
| 12 | 10.6 | 250 | 2980 | 1080 | 680 | 23 | 280 | 101 | 64 |
| 16 | 10.7 | 325 | 2870 | 1410 | 1110 | 30 | 268 | 132 | 104 |

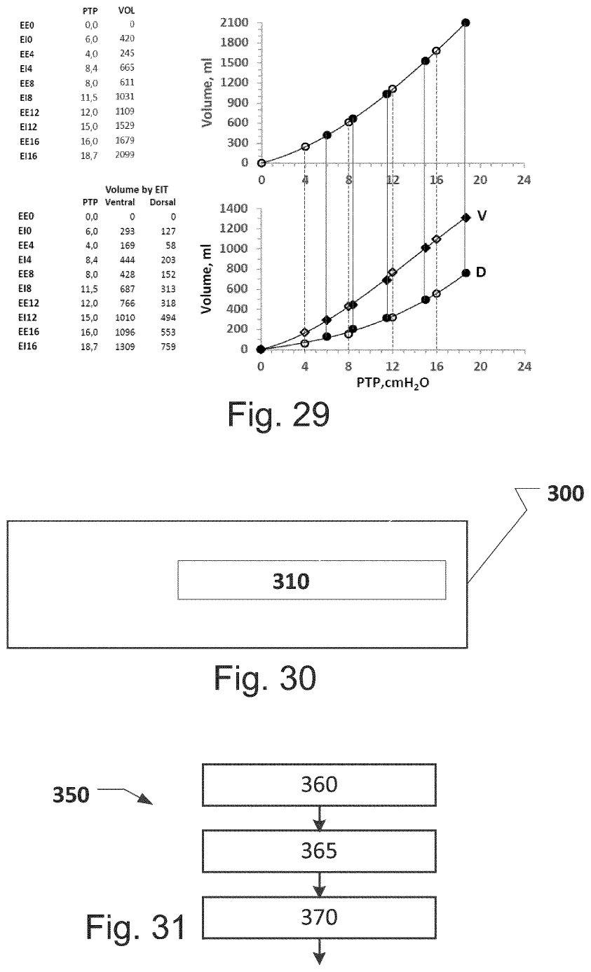

With reference to FIG. 29: Lung P/V curves in a PEEP responder. Upper panels: Whole lung. Lower panels: Lung P/V curves in ventral and dorsal lung. End-expiratory (open circles) and end-inspiratory (closed circles) transpulmonary P/V points. Dashed vertical lines connect end-expiratory P/V points and whole vertical lines connect end-inspiratory transpulmonary P/V points in the whole lung and ventral and dorsal lung separately, as static pressures are the same everywhere

The present invention has been described above with reference to specific embodiments. However, other embodiments than the above described are equally possible within the scope of the invention.

Different method steps than those described above, performing the method by hardware or software, may be provided within the scope of the invention. The different features and steps of the invention may be combined in other combinations than those described. The scope of the invention is only limited by the appended patent claims.

1. A method for determination of End inspiratory transpulmonary pressure (EitP) in a method of stepwise increased positive end-expiratory pressure (PEEP) level(s), wherein said determination is performed within a number of breathing cycles after an increased PEEP level, said number of breathing cycles being less than 5.

2.-19. (canceled)

20. The method of claim 1, wherein a time of said determination is less than 40 seconds.

21. The method of claim 1, wherein said number of breathing cycles is less than 3.

22. The method of claim 1, performed during mechanical ventilation of a patient in volume control mode of a breathing device, wherein the breathing device delivers a double baseline tidal volume before PEEP is increased during ensuing expiration.

23. The method of claim 1, performed during mechanical ventilation of a patient in pressure control mode of a breathing device, wherein the breathing device delivers a tidal volume with the sum of the baseline driving pressure and the increase in PEEP.

24. The method of claim 1, wherein the determination includes providing a tidal volume with a driving pressure at physiological levels, with a plateau pressure below 45 cm H2O.

25. The method of claim 24, wherein the driving pressure is more than 1.3 times a baseline driving pressure before increasing PEEP to the new level.

26. The method of claim 25, wherein the driving pressure is equal to or more than 2.0 times the baseline driving pressure before increasing PEEP.

27. The method of claim 1, performed at regular intervals during mechanical ventilation of a patient.

28. The method of claim 1, comprising a single stepwise increase of said PEEP level.

29. The method of claim 1, comprising multiple stepwise increases of said PEEP level.

30. A breathing apparatus comprising a processing unit configured to determine an End inspiratory transpulmonary pressure (EitP) in a method of stepwise increased positive end-expiratory pressure (PEEP) levels, wherein the determination is performed within a number of breathing cycles after an increased PEEP level, the number of breathing cycles being less than five.

31. The breathing apparatus of claim 30, wherein said breathing device is programmed to deliver a double baseline tidal volume before PEEP is increased when operating in volume control mode.

32. The breathing apparatus of claim 30, wherein said breathing device is programmed to deliver a tidal volume with the sum of the baseline driving pressure and the increase in PEEP when operating in pressure control mode.

33. A method for determination of End inspiratory transpulmonary pressure (EitP) at a highest PEEP level in a method of stepwise increased PEEP levels, wherein the determination is estimated based on previous PEEP steps including estimation of lung elastance EL at the highest PEEP level.

34. The method of claim 33, wherein End inspiratory transpulmonary pressure at the highest PEEP level is estimated by subtracting APPL obtained by linear extrapolation of APPL of at least one lower PEEP level from the end-inspiratory airway pressure at the highest PEEP level.

35. The method of claim 33, wherein End inspiratory transpulmonary pressure at the highest PEEP level is estimated by extrapolating APPL from lower PEEP levels.

36. The method of claim 33, wherein the estimation includes subtracting APPL obtained by linear extrapolation of APPL of the lower PEEP levels from the end-inspiratory airway pressure at the highest PEEP level.

37. A non-transitory computer-readable storage medium encoded with programming instructions for causing a computerized control unit to perform the method of claim 1.

38. A non-transitory computer-readable storage medium encoded with programming instructions for causing a computerized control unit to perform the method of claim 33.
